(19) United States
(12) Patent Application Publication
(10) Pub. No.: US 2022/0115088 A1
(43) Pub. Date: Apr. 14, 2022

(54) Methods For Identifying Complex Disease Subtypes

(71) Applicant: Northeastern University, Boston, MA (US)

(72) Inventors: Jörg Menche, Boston, MA (US); Albert-László Barabási, Boston, MA (US)

(21) Appl. No.: 17/485,105

(22) Filed: Sep. 24, 2021

Related U.S. Application Data
(63) Continuation of application No. 14/903,422, filed on Jan. 7, 2016, now abandoned.
(60) Provisional application No. 61/843,682, filed on Jul. 8, 2013.

(51) Int. Cl. G16B 20/20; G16B 20/00; C12Q 1/6809; C12Q 1/6837; G16H 50/70; G16B 25/00; G16B 25/10

(52) U.S. Cl. CPC G16B 20/20; G16B 20/00; C12Q 1/6809; G16B 40/00; G16H 50/70; G16B 25/00; G16B 25/10; C12Q 1/6837

(57) ABSTRACT

The present technology relates to methods that determine one or more subgroups of subjects within a population of subjects diagnosed with the same disease. In some embodiments, the methods include determining differential gene expression of at least one subgroup in the population using divisive Shuffling Approach (VIStA). In some embodiments, the method includes determining at least one clinical characteristic of each subgroup and/or determining a significant set of clinical characteristics of the disease order.

### CROSS-REFERENCE TO RELATED APPLICATIONS

This application is a Continuation of U.S. application Ser. No. 14/903,422, filed Jul. 7, 2014, which is the U.S. National Stage of International Application No. PCT/US2014/045571, filed on Jul. 7, 2014, published in English, which claims priority to U.S. Application No. 61/843,682, filed on Jul. 8, 2013. The entire teachings of the above applications are incorporated herein by reference.

### GOVERNMENT SUPPORT

This invention was made with government support under Grant Numbers HL108630 and HG004233 awarded by the National Institutes of Health. The government has certain rights in the invention.

### BACKGROUND

A refined understanding of the clinical heterogeneity of a disease can assist in the understanding of the biological mechanisms underlying the disease or its phenotype. Relating clinical and molecular differences of a disease can define specific subgroups of subjects with the disease, wherein each group benefits from different therapeutic interventions.

### SUMMARY

In one aspect, the present technology is related to methods for determining at least one subgroup of subjects from a population of subjects diagnosed with the same disease. In some embodiments, the method includes: (A) obtaining the population of subjects diagnosed with the same disease; (B) dividing the population of subjects into a first group and a second group; (C) calculating the number of differentially expressed genes between the first group and the second group; (D) exchanging one subject from the first group with one subject from the second group; (E) re-calculating the number of differentially expressed genes between the first group and the second group, wherein the exchange of subjects between the first group and second group is maintained if the number of differentially expressed genes between the first group and the second group increases, and wherein the exchange of subjects is rejected if the number of differentially expressed genes between the first group and the second group remains the same or decreases; and (F) repeating steps D-E.

In some embodiments, the method also includes identifying at least one clinical characteristic of the disease, wherein the at least one clinical characteristic is statistically significantly different between the first group and second group.

In some embodiments, the division of the population of subjects is random.

In some embodiments, the determination or re-determination of the number of differentially expressed genes between the first group and the second group is determined by Significant Analysis of Microarrays (SAM), p-values of simple t-tests, Mann-Whitney U-test, Analysis of Variance (ANOVA), and minimal fold change.

In some embodiments, the statistically significant difference of the at least one clinical characteristic is determined by a Mann-Whitney U-test, Fisher's exact test, t-test, Analysis of Variance (ANOVA), chi-square test, Kolmogorov-Smirnov test.

In some embodiments, the statistical significant difference is measured as p≤0.05.

In some embodiments, steps D-E are performed about 1000 times.

In another aspect, the present technology is related to methods for identifying at least one subgroup of subjects from a population of subjects diagnosed with the same disease. In some embodiments, the method includes: (A) obtaining the population of subjects diagnosed with the same disease; (B) dividing the population of subjects into a first group, a second group, and a third group; (C) determining the number of differentially expressed genes between the first group and the second group; (D) exchanging one subject from the first group with a first subject from the third group and one subject from the second group with a second subject from the third group; (E) re-determining the number of differentially expressed genes between the first group and the second group, wherein the exchange of subjects between the first group, second group, and third group are maintained if the number of differentially expressed genes between the first group and the second group increases, and wherein the exchange of subjects between the first group, second group, and third group are rejected if the number of differentially expressed genes between the first group and the second group remains the same or decreases; and (F) repeating steps D-E.

In some embodiments, the method also includes identifying at least one clinical characteristic of the disease, wherein the at least one clinical characteristic is statistically significantly different between the first group and second group.

In some embodiments, the division of the population of subjects is random.

In some embodiments, the determination or re-determination of the number of differentially expressed genes between the first group and the second group is determined by Significant Analysis of Microarrays (SAM), p-values of simple t-tests, Mann-Whitney U-test, Analysis of Variance (ANOVA), and minimal fold change.

In some embodiments, the statistically significant difference of the at least one clinical characteristic is determined by a Mann-Whitney U-test, Fisher's exact test, t-test, Analysis of Variance (ANOVA), chi-square test, Kolmogorov-Smirnov test.

In some embodiments, the statistical significant difference is measured as p≤0.05.

In some embodiments, steps D-E are performed about 1000 times.

In another aspect, the present technology is related to methods for identifying one or more clinical characteristics that identify a subgroup of subjects from a population of subjects diagnosed with the same disease. In some embodiments, the method includes: (A) obtaining the population of subjects diagnosed with the same disease; (B) dividing the population of subjects into a first group and a second group; (C) determining the number differentially expressed genes between the first group and the second group; (D) exchanging one subject from the first group with one subject from the second group; (E) re-determining the number of differentially expressed genes between the first group and the second group, wherein the exchange of subjects between the first group and second group is maintained if the number of differentially expressed genes between the first group and the second group increases, and wherein the exchange of subjects is rejected if the number of differentially expressed genes between the first group and the second group remains the same or decreases; (F) repeating steps D-E; and (G) identifying one or more clinical characteristics of the disease, wherein the clinical characteristics are statistically significantly different between the first group and second group.

In some embodiments, the division of the population of subjects is random.

In some embodiments, the determination or re-determination of the number of differentially expressed genes between the first group and the second group is determined by Significant Analysis of Microarrays (SAM), p-values of simple t-tests, Mann-Whitney U-test, Analysis of Variance (ANOVA), and minimal fold change.

In some embodiments, the statistically significant difference of the at least one clinical characteristic is determined by a Mann-Whitney U-test, Fisher's exact test, t-test, Analysis of Variance (ANOVA), chi-square test, Kolmogorov-Smirnov test.

In some embodiments, the any of the above methods are used with a population of subjects diagnosed with chronic obstructive pulmonary disease (COPD). In some embodiments, the clinical characteristics of the disease in any of the above methods are selected from the group consisting of chronic bronchitis, history of exacerbations, airflow limitation severity (GOLDCD), emphysema quantified by density mask analysis (FV950) or assessed qualitatively by a radiologist (EMPHETCD), body mass index (BMI), phlegm (PHLEGM), age (AGE), 6 minute distance walk (DWALK), cough (COUGH), and sex (SEX).

### BRIEF DESCRIPTION OF DRAWINGS

FIG. 1 is a chart showing clinical and laboratory measurements of the subjects obtained from the ECLIPSE cohort.

FIG. 2A is an exemplary, non-limiting schematic representation of the divisive Shuffling Approach (VIStA).

FIG. 2B is a graph showing the exchanges of 20 exemplary independent VIStA assays.

FIG. 2C is an exemplary non-limiting chart showing how to compare and identify at least one statistically significant clinical characteristic between two groups with maximized differential gene expression.

FIG. 2D provides charts showing an exemplary, non-limiting example of determining at least one statistically significant clinical characteristic between two groups with maximized differential gene expression in 500 independent VIStA assays.

FIG. 3A is a chart showing the number of times a clinical characteristic or inflammatory biomarker were found significantly different between Group 1 and Group 2 in a total of 500 independent VIStA assays.

FIG. 3B is a diagram showing the summary of the independent and pairwise number of significant occurrences of the clinical characteristics. Node size is proportional to the number of times a measure was found significant and the width of a link indicates how often two measures appeared significant in the same VIStA division.

FIG. 3C is a chart showing the number of times that pairwise combinations of clinical characteristics co-occurred in the 500 VIStA outcomes.

FIG. 3D is a chart showing frequent and significant quadruple combinations of GOLDCD, EMPHETCD, and FV950, measuring chronic obstructive pulmonary disease (COPD) severity.

FIG. 3E is a chart showing frequent and significant triplet combinations of GOLDCD, EMPHETCD, FV950, and one selected from the group consisting of measuring BMI, PHLEGM, DWALK and AGE, measuring COPD severity.

FIG. 4 is a chart summarizing non-limiting, exemplary clinical characteristics of COPD subjects as identified by clinical experts.

FIG. 5 is a chart summarizing clinical measures, biomarkers, and cell counts among the four groups of COPD patients identified from the results of FIG. 3.

FIG. 6A is a Venn diagram showing the combinations of phenotypic measures that define the subtypes predicted by the VIStA method.

FIG. 6B is a Venn diagram showing the number of differentially expressed genes unique to each subtype, as well as common to all four subtypes.

FIG. 6C is a Venn diagram that shows that the common genes show a large overlap with the genes differentially expressed between subjects with GOLDCD 2 and subjects with GOLDCD 3&4.

FIG. 7A is a chart showing the top common pathways among Common Genes, Group 1 genes and Group II genes from FIG. 5.

FIG. 7B is a chart showing the top common pathways among Group III and Group IV genes from FIG. 5.

FIG. 8 is a chart showing the top ten up regulated and down regulated genes and their fold-change (FC) in each group (in group II, only five genes were down regulated).

### DETAILED DESCRIPTION

It is to be appreciated that certain aspects, modes, embodiments, variations and features of the technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a”, “an” and “the” include plural references unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein “clinical characteristic” refers to clinical signs and symptoms of a disease or disorder. Clinical characteristics of specific disease and disorders are known in the art. For example, clinical characteristics of chronic obstructive pulmonary disease include, but are not limited to, airflow limitation severity (GOLDCD), emphysema quantified by density mask analysis (FV950) or assessed qualitatively by a radiologist (EMPHETCD), body mass index (BMI), phlegm (PHLEGM), age (AGE), 6 minute distance walk (DWALK), cough (COUGH), and sex (SEX).

As used herein, “differentially expressed gene” refers to a gene whose expression level in one group shows a statistically significant difference compared to the expression level of the same gene in another group.

As used herein “a heterogeneous disease or disorder” refers to a disease or disorder that comprising multiple different subtypes.

As used herein “independent VIStA assay” refers to performing the VIStA method on a population of subjects to identify at least one group or subgroup, e.g., a first subgroup of subjects, with a significant difference in its gene expression as compared to at least one other subgroup of subjects, e.g., a second, third, or fourth subgroup, from the population of subject. In some embodiments, more than one independent VIStA assay is performed on the same population of subject.

As used herein “statistically significant” or “significant” refer to a statistical analysis that results in a p value that is less than or equal to 0.05, i.e., p≤0.05, or less than or equal to 0.01, i.e., p≤0.01, or less than or equal to 0.001, i.e., p≤0.001. A skilled artisan would be able to determine the appropriate p value based on the statistical analysis being performed.

The present technology relates to methods and systems for classifying subjects into at least two distinct groups, e.g., subgroups, from a population of subjects. In some embodiments, the diVIsive Shuffling Approach (VIStA) method is used to identify groups with systematic statistical differences by randomly exchanging group members. In some embodiments, VIStA is used to identify subgroups within a population according to, but not limited to, e.g., gene expression, DNA methylation, expression quantitative trait loci (eQTLs), and single nucleotide polymorphisms (SNPs).

In some embodiments, the population of subjects is diagnosed with the same disease. In some embodiments, the subjects in the population are separated into distinct groups based on gene expression profiles and/or one or more clinical characteristics exhibited by the subjects. In some embodiments, VIStA is used to identify groups of subjects by measuring differences in gene expression, as a function of the number of differentially expressed genes between the groups. In some embodiments, the groups classified by VIStA are used to identify clinical parameters showing significant clinical characteristics between the groups.

The VIStA approach is fundamentally different from clustering techniques like hierarchical or k-means clustering. The latter attempt to identify cohesive groups based on similarity, while VIStA is a method based on maximizing the differences between groups. Another important difference to standard clustering approaches is that VIStA is able to identify a large number of locally optimal divisions.

### Determining Groups of Subjects Based on Differential Gene Expression Using VIStA

In one aspect, the present technology relates to methods for determining at least one group, e.g., a subgroup, of subjects from a population of subjects. In some embodiments, the population of subjects is diagnosed with the same disease or disorder. In some embodiments, a first subgroup has a “statistically significant” difference in the number of differentially expressed genes as compared to the rest of the population of subjects or to another group of subjects from the population, e.g., a second subgroup.

In some embodiments, VIStA is used to identify at least one group of subjects with a pattern of differentially expressed genes within a population of subjects. In some embodiments, the population of subjects is diagnosed with the same disease or disorder.

In some embodiments, VIStA includes the steps of:

(A) obtaining a population of subjects diagnosed with the same disease,

(B) dividing the population into a first group, e.g., a first subgroup, and a second group, e.g., a second subgroup,

(C) determining the number of differentially expressed genes between the first group and the second group,

(D) exchanging one subject from the first group with one subject from the second group, (E) re-determining the number of differentially expressed genes between the first group and the second group, wherein the exchange of subjects between the first group and second group is maintained if the number of differentially expressed genes between the first group and the second group increases, and wherein the exchange of subjects is rejected if the number of differentially expressed genes between the first group and the second group remains the same or decreases, and

(F) repeating steps D-E.

In another embodiment, VIStA includes the steps of:

(A) obtaining a population of subjects diagnosed with the same disease,

(B) dividing the population into a first group, e.g., a first subgroup, and a second group, e.g., a second subgroup, and third group e.g., a third subgroup or a reservoir,

(C) determining the number of differentially expressed genes between the first group and the second group,

(D) exchanging one subject from the first group with one subject from the third group and one subject from the second group with one subject from the third group;

(E) re-determining the number of differentially expressed genes between the first group and the second group, wherein the exchanges of subjects between the first group and third group and the second group and the third group are maintained if the number of differentially expressed genes between the first group and the second group increases, and wherein the exchange of subjects between the first group, second group, and third group are rejected if the number of differentially expressed genes between the first group and the second group remains the same or decreases, and

(F) repeating steps D-E.

In some embodiments, the number of subjects in the population is between about 10 to 100, between about 20 to 90, between about 30 to 80, between about 40 to 70, or between about 50 to 60. In some embodiment, the number of subjects in the population of subjects diagnosed with the same disease or disorder is between about 100 to 1000, between about 200 to 900, between about 300 to 800, between about 400 to 700, or between about 500 to 600. In some embodiments, the number of subjects in the population of subjects diagnosed with the same disease or disorder is between about 1000 to 10,000, between about 2000 to 9000, between about 3000 to 8000, between about 4000 to 7000, or between about 5000 to 6000.

The disease or disorder of the population of subjects can be any disease or disorder. By way of example, but not by way of limitation, in some embodiments, the disease or disorder includes, but is not limited to, chronic obstructive pulmonary disease (COPD), lung cancer, breast cancer, diabetes (e.g., Type 1 or Type 2 diabetes), asthma, Huntington's disease, Alzheimer's, Parkinson's, and heart disease. In some embodiments, the disease or disorder is a heterogeneous disease or disorder.

The gene expression profile of a subject can be determined by any method known in the art. By way of example, but not by way of limitation, in some embodiments, the gene expression is determined by Northern blotting, reverse transcription polymerase chain reaction (RT-qPCR), Western blot, microarrays, e.g., DNA microarray, single nucleotide polymorphism (SNP) arrays, protein arrays, or a combination thereof. See, e.g., Lashkari et al., Proc. Natl. Acad. Sci. U.S.A., 94(24): 13057-13062 (1997), Singh et al., Thorax, 66(6):489-95 (2011), and Ding et al., J Biomol Tech., 18(5): 321-330 (2007).

By way of example, but not by way of limitation, in some embodiments, a DNA microarray method for determining gene expression includes attaching a plurality of microscopic DNA spots to a solid surface, wherein each DNA spot contains a specific DNA sequence (known as probes, reporters, or oligos), contacting the DNA spots with a sample containing DNA from a subject, hybridizing the DNA in the sample to the DNA spots under hybridization conditions, and detecting and quantifying the hybridization by fluorescence.

In some embodiments, the initial division of the population into groups is random. In some embodiments, the subjects selected to be exchanged between groups is random.

In some embodiments, the determination or re-determination of the number of genes differentially expressed between the groups is determined by a technique selected from the group consisting of Significant Analysis of Microarrays (SAM), p-values of simple t-tests, Mann-Whitney U-test, Analysis of Variance (ANOVA), and minimal fold change.

In some embodiments, the increase in the number of differentially expressed genes between the first group and the second group is at least one gene.

In some embodiments, all genes are measured for differentially gene expression. In some embodiments, only genes related to the disease or disorder are measured for differential expression. Genes related to specific diseases and disorders are known in the art. By way of example, but not by way of limitation, genes related to breast cancer include, but are not limited to, BRAC1, BRAC2, and receptor tyrosine-protein kinase erbB-2 (ERBB2, also known as HER2/neu).

False discovery rate (FDR) is number of false predictions divided by the number of total predications. For example, a FDR of 0.05 means that out of 100 predicted positives, 5 are wrong. In some embodiments, the FDR in SAM is less than or equal to 0.1, i.e., FDR≤0.1. In some embodiments, the FDR in SAM is based on a comparison with random permutations.

In some embodiments, steps D-E are repeated about 500 to 3000, about 750 to 2500, about 1000 to 2000, or about 1250 to 1750 times per independent VIStA assay to produce at least one subgroup, e.g., a first subgroup of subjects, with a significantly different gene expression pattern as compared to at least one other subgroup of subjects, e.g., a second, third, or fourth subgroup. By way of example, but not by way of limitation, in some embodiments of an independent VIStA assay, steps D-E of an independent VIStA assay are repeated 1000 times.

The use of VIStA to determine groups of subjects is not intended to be limited to use with differential gene expression. VIStA is useful for determining groups or subgroups of a population based on, e.g., differential DNA methylation, differential expression of quantitative trait loci (eQTLs), and/or differential expression of single nucleotide polymorphisms (SNPs).

Any method known in the art for measuring differential DNA methylation can be used. By way of example, but not by way of limitation, in some embodiments, differential DNA methylation is determined by Quantitative Differentially Methylated Regions (QDMR) method.

Any method known in the art for measuring differential expression of quantitative trait loci (QTL) can be used. By way of example, but not by way of limitation, in some embodiments, differential expression of QTL is determined by QTL mapping.

Any method known in the art for measuring differential expression of SNPs can be used. By way of example, but not by way of limitation, in some embodiments, differential expression of SNPs is determined by QualitySNP.

In some embodiments, a statistically significant difference in the level of at least one: differentially methylated DNA, differentially expressed QTL, or differentially expressed SNP between a first group and a second group results in a maintained exchange in the VIStA assay.

### Methods for Determining Clinical Characteristics of Diseases or Disorders Based on Groups Determined by VIStA

In some embodiments, at least one significantly different clinical characteristic is identified between at least two groups/subgroups that were determined by an independent VIStA assay. In some embodiments, the method for identifying at least one significantly different clinical characteristic includes performing one or more independent VIStA assays on a population of subjects to identify at least one group that have a significant number of differentially expressed genes as compared to at least one other group and analyzing at least two groups for statistically significant clinical characteristics.

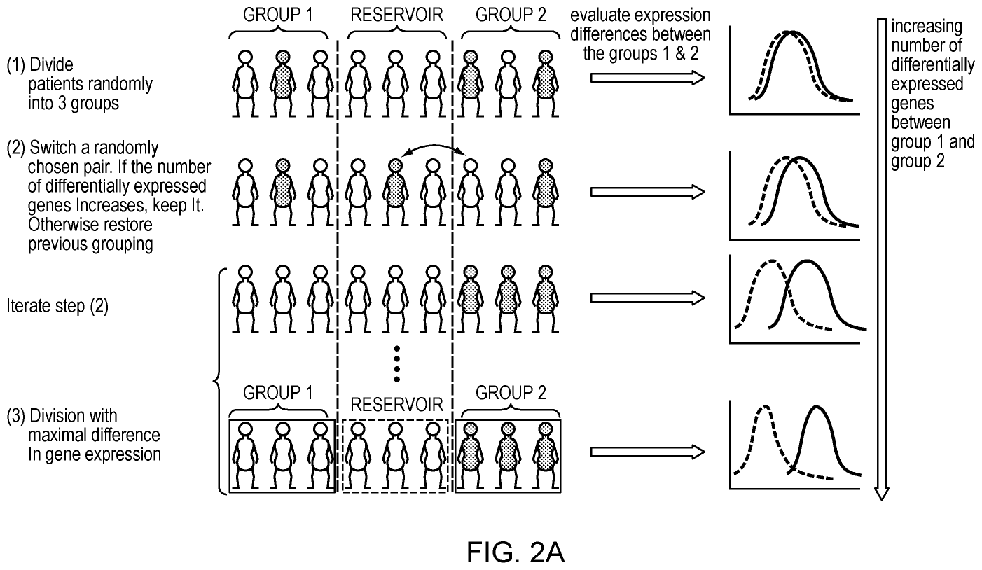

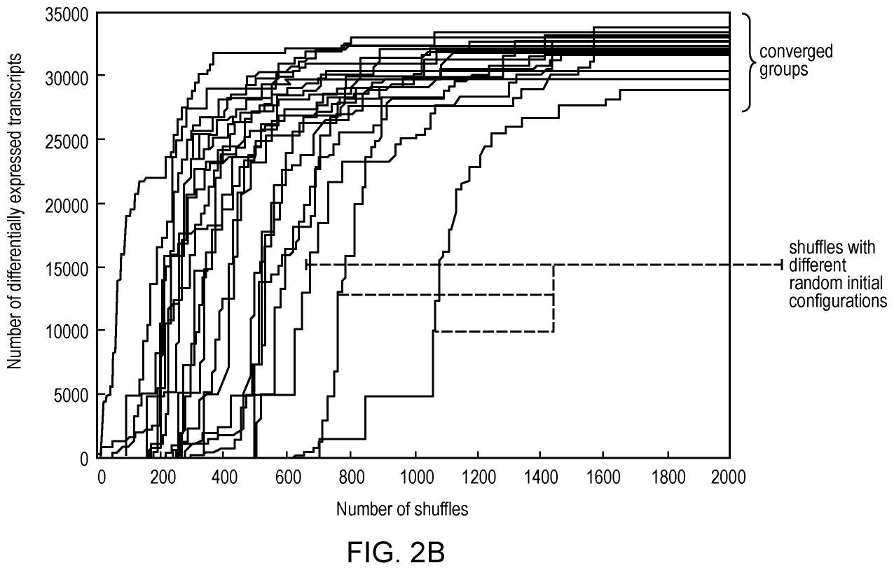

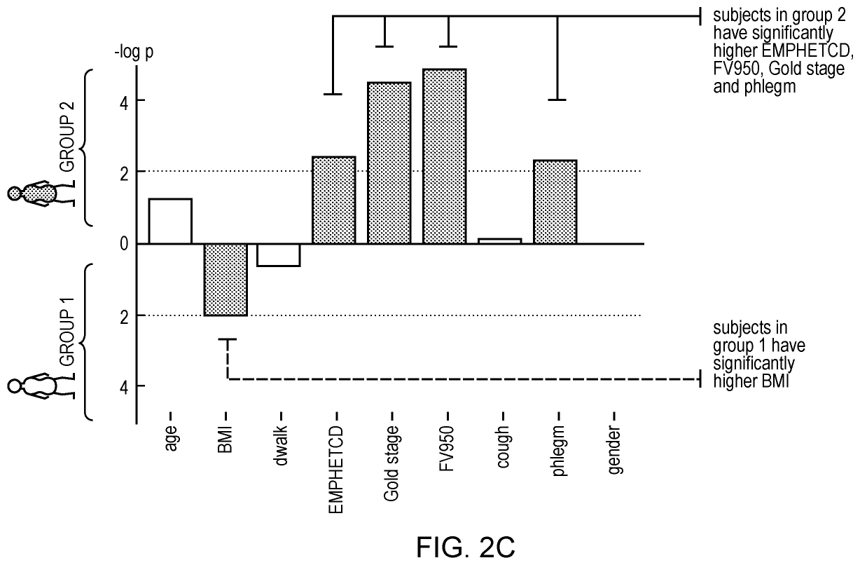

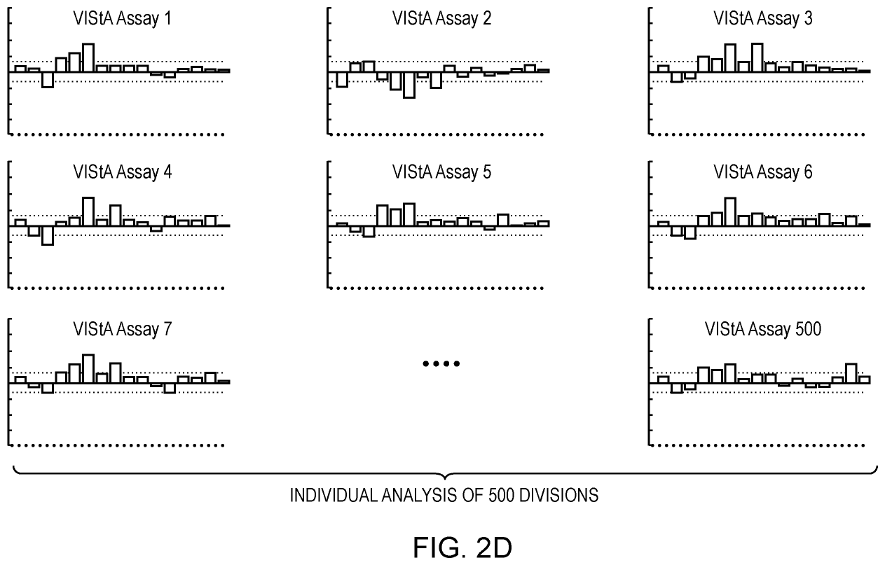

FIG. 2C is an exemplary graph for identifying statistically significant clinical characteristics between two groups determined by an independent VIStA to have maximized differential gene expression. By way of example, but not by way of limitation, FIG. 2C lists some clinical characteristics of COPD and shows which clinical characteristics are statistically significant, e.g., BMI, EMPHETCD, GOLD stage, and phlegm, and which group express said significant clinical characteristics.

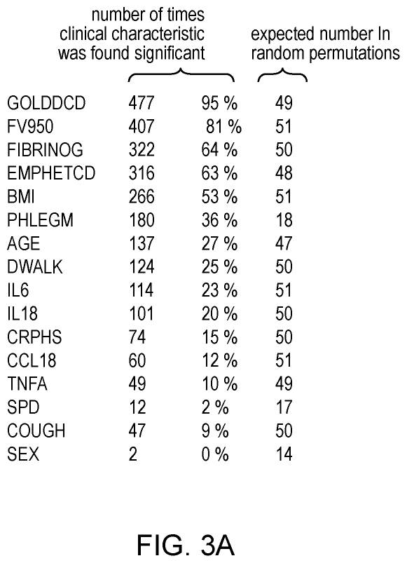

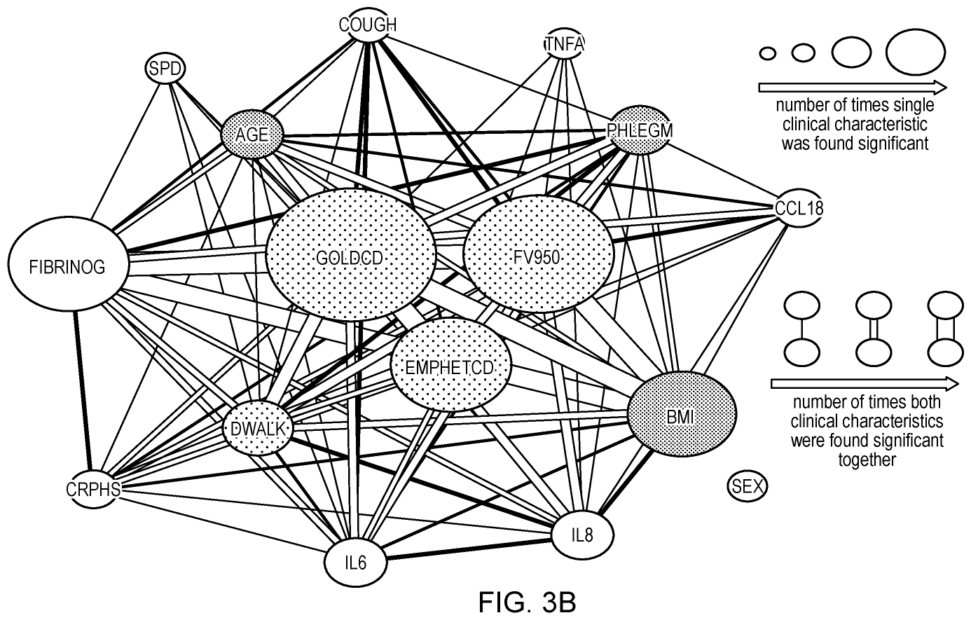

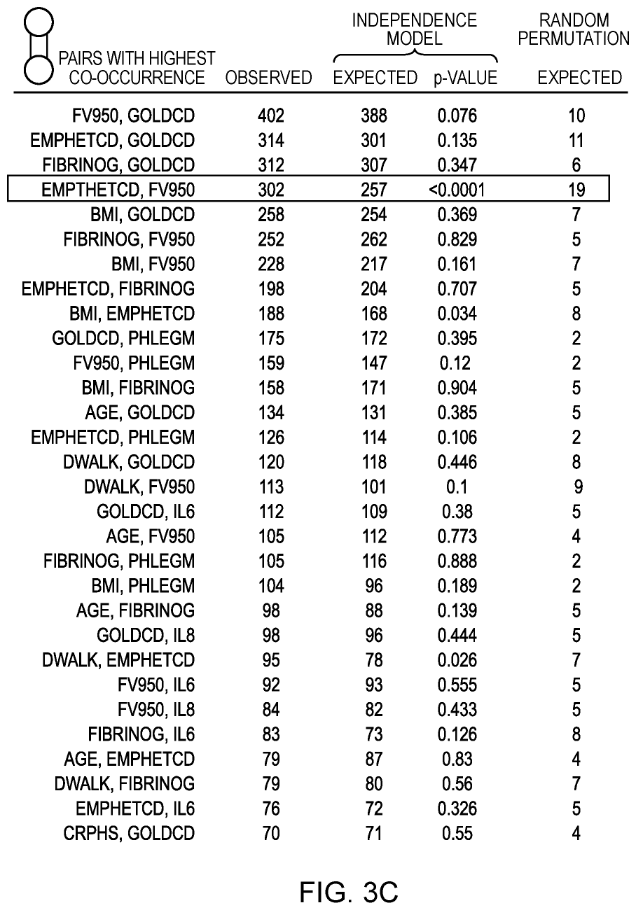

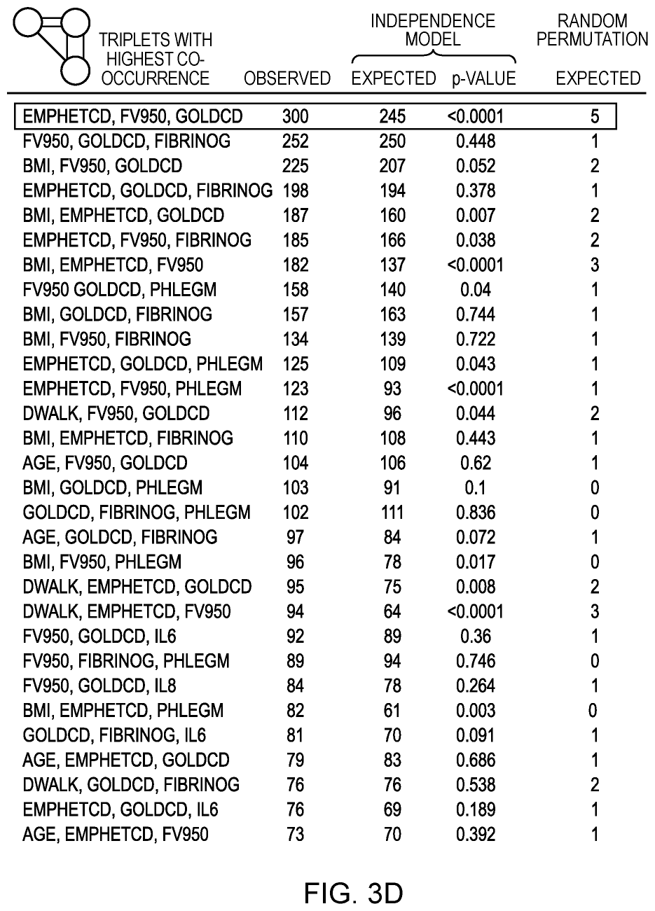

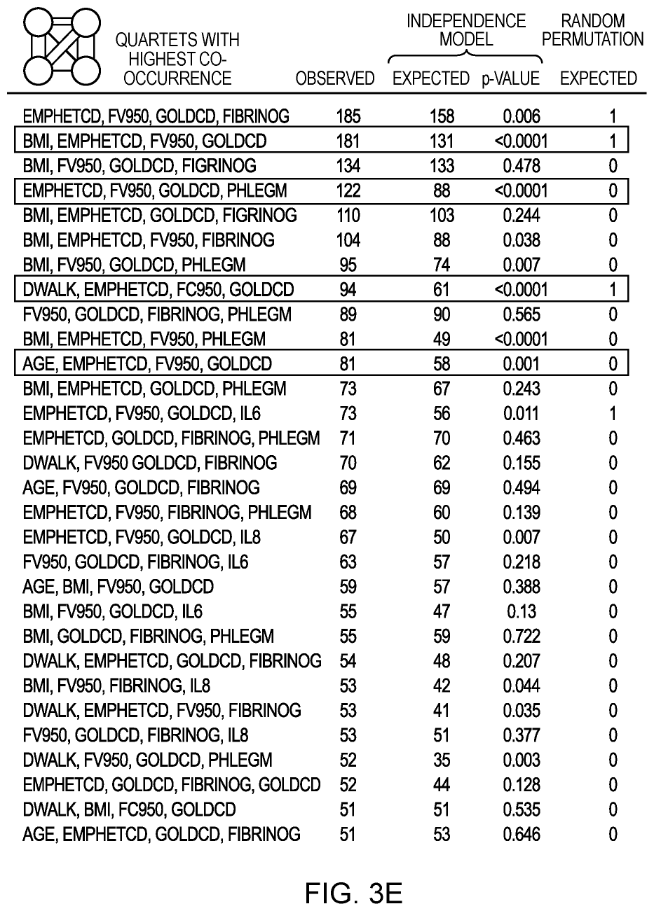

In some embodiments, analysis of the clinical characteristics of the population of subjects includes performing more than one independent VIStA assay with the population of subjects and analyzing the collective clinical characteristics to identify a set of significant clinical characteristics. FIG. 2D shows exemplary graphs of eight independent VIStA assays (independent VIStA assays 1-7 and 500) and FIGS. 3A-3B show the collective clinical characteristic's data from 500 independent VIStA assays.

In some embodiments, an independent VIStA assay using the same subject population is performed between about 1 to 1000 times, between about 100 to 900 times, between about 200 to 800 times, between about 300 to 700 times, between about 400 to 600 times, or between about 450 to 550 times. In some embodiments, each independent ViStA assay performed is analyzed for statistically significant different clinical characteristics (see FIG. 2C-2D as an example).

In some embodiments, each initial division of the subject population for each independent VIStA assay is random and/or non-identical to other independent VIStA assay of the same population.

In some embodiments, statistically significant different clinical characteristics are determined by the Mann-Whitney U-test, Fisher's exact test, t-test, Analysis of Variance (ANOVA), chi-square test, Kolmogorov-Smimov test. In some embodiments, the significance threshold of a significance test is p≤0.05 or 0.01. Based on the significance test being performed and the data being analyzed, a skill artisan would be able to determine the significance threshold.

Clinical characteristics assayed for significance can be any clinical characteristic known in the art for a particular disease or disorder. In some embodiments, the disease or disorder is a heterogeneous disease or disorder. By way of example, but not by way of limitation, in some embodiments, clinical characteristics include, but are not limited to, airflow limitation severity (GOLDCD), emphysema quantified by density mask analysis (FV950) or assessed qualitatively by a radiologist (EMPHETCD), body mass index (BMI), phlegm (PHLEGM), age (AGE), 6 minute distance walk (DWALK), cough (COUGH), and sex (SEX).

In some embodiments, statistically significant different inflammatory biomarker levels are analyzed between at least two groups determined by VIStA. Inflammatory biomarker include, but are not limited to, interleukin-6 (IL-6), IL-8, high-sensitivity C-reactive protein (HSCRP), chemokine motif (C-C) ligand 18 (CCL18), surfactant protein D (SPD), fibrinogen (FIBRINOG), and tumor necrosis factor alpha (TNFA).

In some embodiments, combinations of one or more statistically significant clinical characteristics and/or statistically significant inflammatory biomarkers from one or more independent VIStA assays are compared and/or analyzed. By way of example, but not by way of limitation, in some embodiments, statistical significance of a pairwise co-occurrence of statistically significant clinical characteristics between two subgroups is calculated using a binomial model that assumes independence of the individual characteristics or biomarker levels as the Null hypothesis. In some embodiments, the significant clinical characteristics identified are used to identify subjects or groups of subjects for evaluating therapeutic agents or methods of treatments.

In some embodiments, statistically significant different clinical characteristics from one or more subgroups from a first independent VIStA assay are compared to statistically significant different clinical characteristics from one or more subgroups from a second independent VIStA assay. In some embodiments, statistically significant different clinical characteristics from 2, 3, 4, 5, 6, 7, 8, 9, 10 or more subgroups from an array of independent VIStA assays, e.g., 500 independent VIStA assays, are compared.

In some embodiments, two or more groups/subgroups determined from VIStA are analyzed for a core set of shared genes within the groups/subgroups. Additionally, or alternatively, in some embodiments, the up regulation and/or down regulation of genes (e.g., as determined by gene expression level) and/or the fold change of gene expression between two or more groups determined from VIStA are analyzed. Additionally, or alternatively, in some embodiments, two or more groups/subgroups determined from VIStA are analyzed for one or more pathways shared by the groups.

In some embodiments, identification of significant clinical characteristics, core sets of shared gene expression profiles, up and down regulated genes, and activated pathways from subgroups determined by VIStA are useful for, but not limited to, e.g., defining novel subtypes of a disease or disorder, accurately diagnosing a patient, and identifying targeted therapy for patients or subgroups of patients, e.g., personalized medicine.

Without wishing to be bound by theory, identification of significant clinical characteristics based on VIStA is useful as the identified groups are based on molecular differences. Accordingly there is a link between the low-level molecular characteristics of subjects and their high-level clinical characteristics. The identified link is useful for developing effective design of therapeutic treatments.

### EXAMPLES

The following examples are provided to more fully illustrate various implementations of the present technology. These examples should in no way be construed as limiting the scope of the present technology.

### Example 1: Identifying Significant Clinical Characteristics of Chronic Obstructive Pulmonary Disease (COPD) Using VIStA

This example shows the use of VIStA to determine subgroups of COPD subjects from a population of COPD subjects.

Methods

The ECLIPSE COPD cohort is a large, prospective, observational and controlled study (Clinicaltrials.gov identifierNCT00292552; GSK study code SCO104960), whose design has been previously published. See Vestbo et al., The European respiratory journal: official journal of the European Society for Clinical Respiratory Physiology, 31(4):869-73 (2008). Briefly, the ECLIPSE COPD cohort was a 3 year observational, international, multicenter study that collected clinical, genetic, proteomic, and biomarker data in a population of COPD subjects.

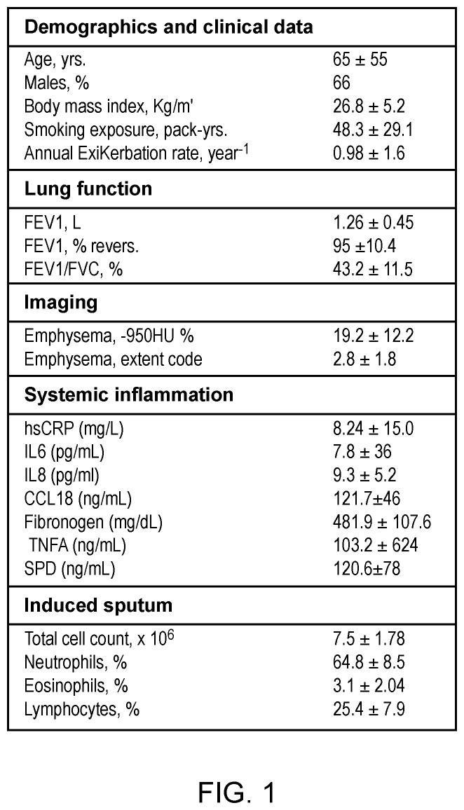

Gene expression data from induced sputum samples from 140 former smokers from the ECLIPSE study (70 with moderate or airflow limitation (GOLDCD) stage 2 and 70 with severe or GOLDCD stage 3-4 airflow limitation, matched for age and gender) were analyzed for differential gene expression. Characteristic of the 140 subjects are disclosed in FIG. 1. Sputum induction and processing with dithiothreitol (DTT) was performed using standard methods as previously described in DeMeo et al., Proceedings of the American Thoracic Society, 3(6):502 (2006). Generation and processing of gene expression data was performed as described in Singh et al., Thorax, 66(6):489-95 (2011).

An independent VIStA was performed by randomly dividing the 140 subjects into three groups, Groups 1-2 and a reservoir (Group 3), see FIG. 2A. Groups 1 and 2 had 50 subjects each and the reservoir had 40 subjects. The number of differentially expressed genes between Group 1 and 2 was determined by Significant Analysis of Microarrays (SAM) with FCR≤0.1. After the initial number of differentially expressed genes between Group 1 and 2 was measured, a random member of Group 1 was exchanged with a random member from the reservoir and a random member of Group 2 was exchanged with a random member from the reservoir. After the exchanges, the number of differentially expressed genes between Group 1 and 2 was re-determined by SAM. If the number of differentially expressed genes between Group 1 and 2 increased, then the exchange was maintained and if the number of differentially expressed genes between Group 1 and 2 decreased or stayed the same, then the exchange was rejected and the exchanged was reversed. The random exchange of members of Group 1 and Group 2 with the reservoir and determination of the number of differentially expressed genes between Group 1 and 2 was repeated 2000 times. FIG. 2B.

Five hundred independent VIStA assays were performed. FIG. 2B is an exemplary sample of twenty independent VIStA assays from the five hundred independent VIStA assays.

Results

FIG. 2B shows that the exchange of subjects between Groups 1 and 2 with the reservoir eventually leads to two groups with a maximum number of differentially expressed genes.

These results shows that VIStA is useful for determining one or more subgroups with differentially expressed genes from a population of subjects diagnosed with the same disease.

### Example 2: Identification of Significant Clinical Characteristics of COPD Based on VIStA

This example shows that subgroups determined by the VIStA assay can be used to determine significant clinical characteristics of COPD.

Methods

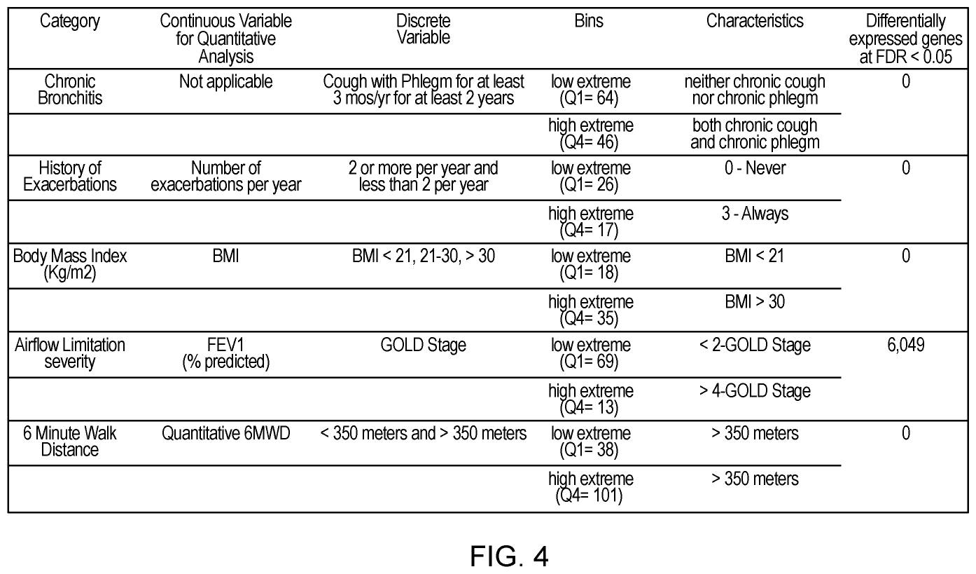

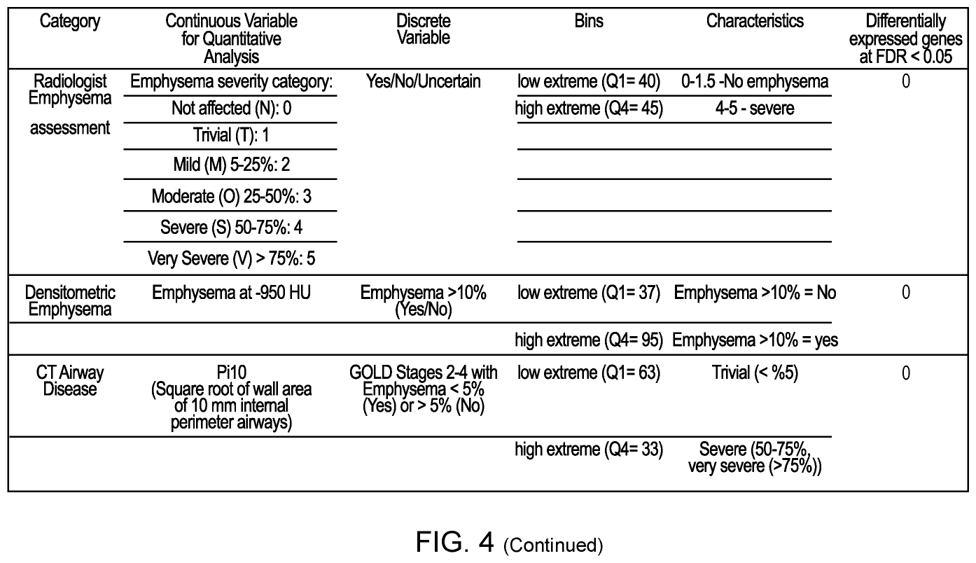

Control Assay: The control assay identified statistically significant gene expression differences between patient groups that differ in a single clinical characteristic. For each of the COPD clinical characteristics of: chronic bronchitis, history of exacerbations, body mass index, airflow limitation severity, 6 minute walk distance, radiologist emphysema assessment, densitometric emphysema, and CT airway disease, the 140 subjects were divided into two groups based on the clinically relevant cut-points (see FIG. 4, column 5), e.g., for chronic bronchitis the subjects were divided by neither chronic cough or phlegm and both chronic cough and chronic phlegm. Gene expression analysis was performed using Significance Analysis of Microarrays (SAM) with a false discovery rate (FDR) of 5% (FDR<0.05).

VIStA Assay: Five hundred independent VIStA assays were performed, as described in Example 1, wherein each independent VIStA assays began with a different random initial 3-group (2 groups and one reservoir) configuration. Each of the 500 pairs of groups, i.e., Groups 1 and 2 resulting from each VIStA assay performed, were analyzed for statistically significant clinical characteristics and inflammatory biomarkers between the two groups, see FIGS. 2C and 2D and FIG. 3. The COPD clinical characteristics analyzed included airflow limitation severity (GOLDCD), emphysema quantified by density mask analysis (FV950) or assessed qualitatively by a radiologist (EMPHETCD), body mass index (BMI), phlegm (PHLEGM), age (AGE), 6 minute distance walk (DWALK), cough (COUGH), sex (SEX), see FIG. 3A. Inflammatory biomarker levels analyzed included interleukin-6 (IL-6), IL-8, high-sensitivity C-reactive protein (HSCRP), chemokine motif (C-C) ligand 18 (CCL18), surfactant protein D (SPD), fibrinogen (FIBRINOG), and tumor necrosis factor alpha (TNFA), see FIG. 3A. Significance of the clinical characteristics and inflammatory biomarkers between Groups 1 and 2 was determined by using Mann-Whitney U-test (significance threshold of p≤0.05) for all continuous characteristics, (e.g., BMI) and Fisher's exact test for binary characteristics (e.g., gender).

Results

Control Assay: As shown in FIG. 4, column 6, apart from the severity of airflow limitation as assessed by the GOLD stage, none of the other clinical measures identified significant gene expression changes. This failure suggests that these clinical characteristics are not sufficiently discriminative to capture gene expression variation in COPD.

VIStA Assay: FIG. 3A shows that the severity of airflow limitation (GOLDCD) was the single most important determinant of differential gene expression, being statistically significant in 95% of all independent VIStA outputs (n=477). The second most common clinical determinant of differential sputum gene expression was emphysema, quantified by either density mask analysis (FV950) or assessed qualitatively by the radiologist (EMPHETCD) (81% and 63% of all independent VIStA outcomes, respectively, FIG. 3A). BMI, Phlegm, age and DWALK were observed in 53%, 36%, 27% and 25% of all independent VIStA outcomes, respectively (FIG. 3A). Plasma fibrinogen was the most frequently identified systemic biomarker (64% of all independent VIStA outcomes).

These result show that the VIStA assay is an improved method for identifying significant differential gene expression of subgroups in a population of subjects diagnosed with the same disease. Additionally, the VIStA assay is useful for determining correlations between clinical characteristics and gene expression.

### Example 3: Combination of COPD Clinical Traits Based on VIStA

This example shows how subgroups identified by independent VIStA assays can be used to determine a set of significant clinical characteristics.

Methods

To quantify the extent to which the VIStA outcomes could reflect spurious associations, 10,000 random divisions of the patients were generated and analyzed as to how often the individual characteristics and their combinations appear as significant (FIG. 3C-E). The statistical significance of each co-occurrence (FIG. 3C-E) was calculated using a binomial model that assumes independence of the individual characteristics or biomarker levels as the Null hypothesis.

Results

FIG. 3B illustrates how often combinations (pairs) of significant single clinical characteristics (or inflammatory biomarkers) co-occur in the different VIStA assays by the width of the links between them. The VIStA assays show a much higher number of significant clinical characteristics than expected by chance, with the exceptions of the biomarkers CCL18, TNFA and SPD and the variables COUGH and SEX (FIG. 3B).

FIG. 3C shows that the pairwise co-occurrences of clinical characteristics and inflammatory biomarkers were dominated by airflow limitation severity (GOLDCD). Other characteristics frequently observed in combinations include emphysema (EMPHETCD or FV950), fibrinogen levels, phlegm, BMI and age. Most pairs appear with the frequency expected for the Null hypothesis of independent individual clinical characteristics (see the non-significant p-values in FIG. 3C-E), implying that their association is not significant (e.g., EMPHETCD and GOLDCD). A notable exception is EMPHETCD and FV950, whose statistical association is expected, given that the two variables are not independent but are different measures of the same clinical characteristic (emphysema).

FIGS. 4D-E shows the observed and expected co-occurrence of triplets and quartets of clinical characteristics and inflammatory biomarkers. The most frequent and significant triplet consists of severity of airflow limitation (GOLDCD) and the two emphysema measures EMPHETCD and FV950 (FIG. 3D). GOLDCD and either one of the severity of emphysema measures FV950 or EMPHETCD co-occurred in almost all triplets.

FIG. 3E lists the most frequent combinations of four variables. The most significant combinations are those which include the triple GOLDCD, FV950 and EMPHETCD, together with one additional variable, the most significant being FIBRINOGEN, BMI, PHLEGM, DWALK and AGE.

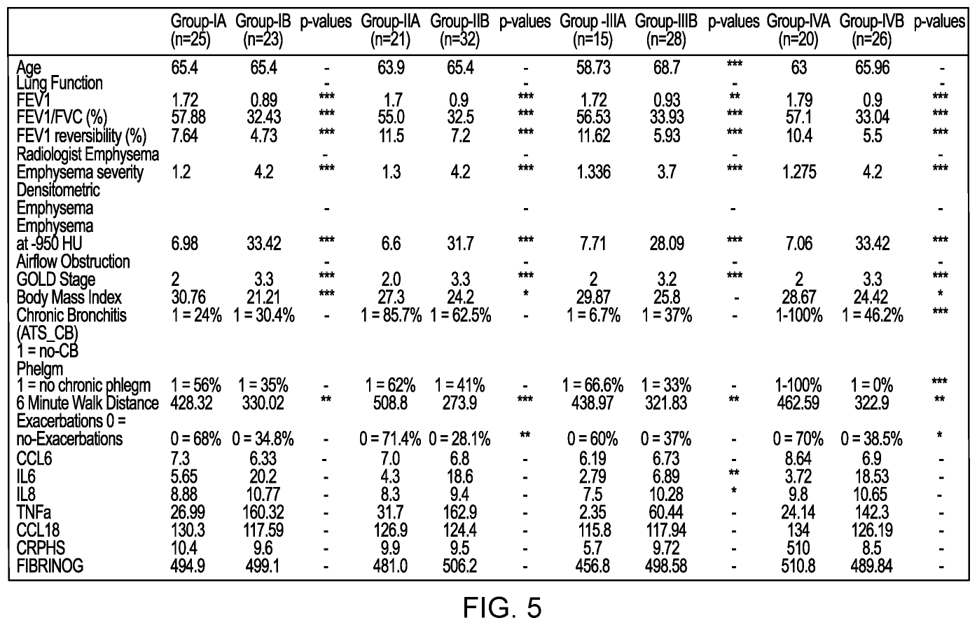

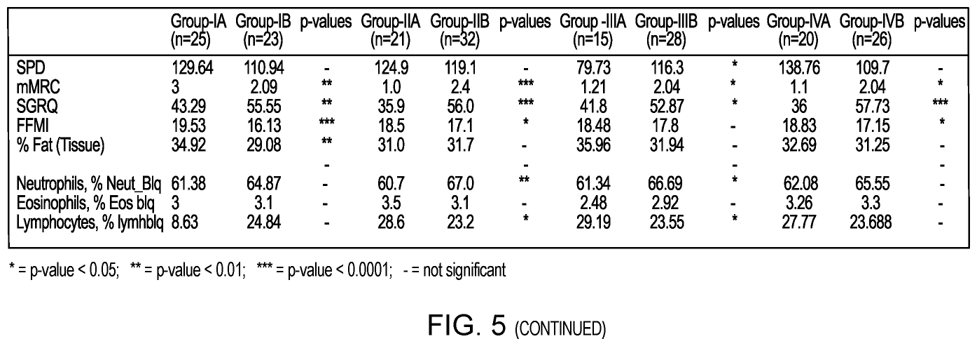

FIGS. 3C-E indicates four distinct clinical parameters that define groups of patients with considerable gene expression differences. In all groups the patients are characterized by different disease severity (GOLDCD) and emphysema (i.e., EMPHETCD and FV950) but in addition, each group also has one clear distinctive parameter: high/low BMI (Group I), exercise capacity (DWALK) (Group II), Age (Group III) or presence/absence of phlegm production (Group TV) (FIG. 5). For example, group TA has high GOLDCD, emphysema, FV950 and low BMI, while group IB has low GOLDCD, emphysema, FV950 and high BMI, see FIG. 5.

To further characterize the subtypes determined by the independent VIStA assays, all subjects were subdivided into groups according to the identified clinical characteristics of GOLDCD, EMPHETCD, FV950, and either BMI (Group I), DWALK (Group II), AGE (Group III) or Phlegm (Group IV), see FIG. 5. First, the number of clinical, biomarker and cell count measures of the subjects in each group was analyzed. An exemplary finding was that serum levels of the biomarkers IL-6, IL-8 and SPD are significantly higher in group III B than in III A, a difference that was not observed in other groups (FIG. 5). Similarly, the proportion of neutrophils and lymphocytes in sputum were significantly higher in group III B in comparison to III A (FIG. 5).

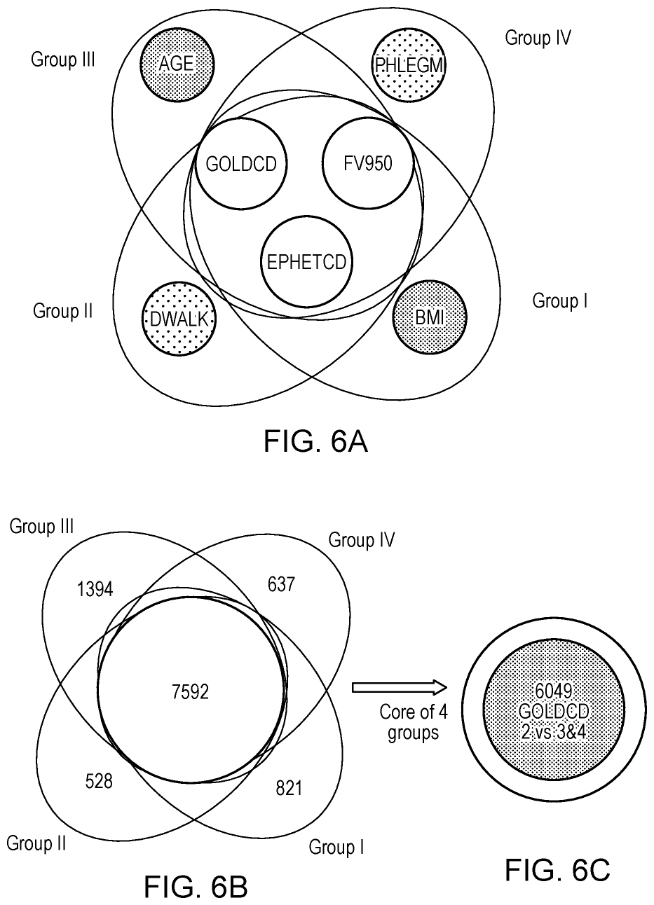

A separate differential gene expression analysis was performed with a FDR<0.05 on the subgroups, finding 821 unique genes for Group I, 528 for Group II, 1,394 genes for Group III and 637 for Group IV (FIG. 6A-B). The four groups shared 7,592 genes that are differentially expressed in all of them. 80% of these genes were previously identified as differentially expressed comparing patients with moderate (GOLD 2) with those with more severe disease (GOLD 3&4) (FIG. 6C). The results indicated that the common core is dominated by severity of COPD, while the uniquely differentially expressed genes between the groups represent additional variation.

These results show that the VIStA assay is useful for determining combinations of clinical characteristics that correlated to gene expression differences.

### Example 4: Specific Genes and Pathways of COPD in the Subgroups from VIStA

This example shows the use of the VIStA assay in a pathway enrichment analysis to determine the core set of genes common to all groups, as well as for the unique gene set of each group.

Method

Pathway annotations were obtained from the Molecular Signatures Database (MSigDB) published by the Broad Institute, Version 3.1, see Subramanian et al., PNAS, 102(43):15545-15550 (2005). MSigDB integrates several different pathway databases, the KEGG, Biocarta and Reactome were used. The enrichment analysis between a given gene set and a pathway was done using Fisher's exact test.

Results

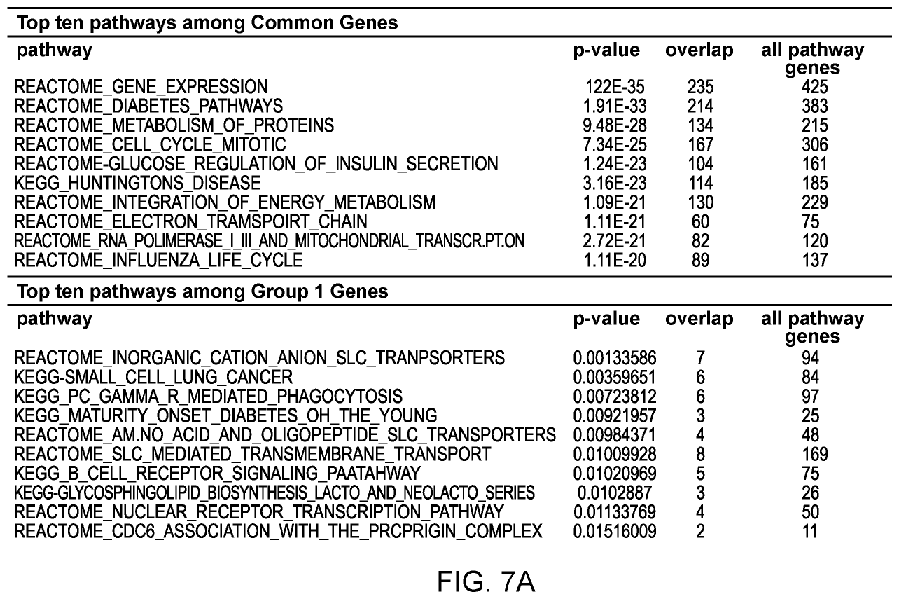

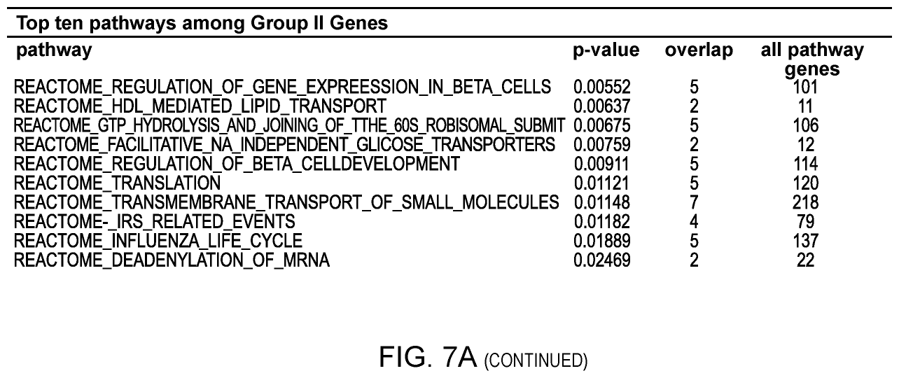

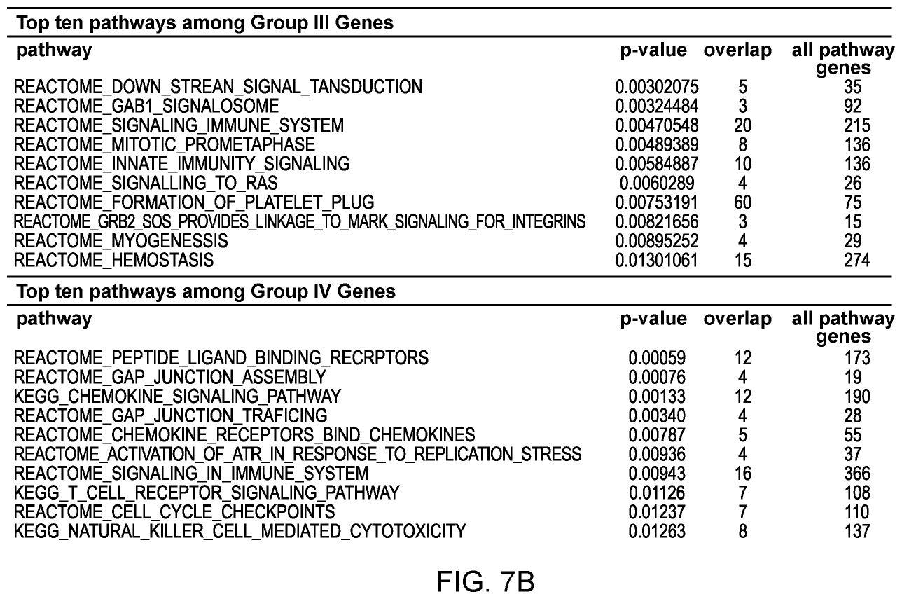

As shown in FIG. 7, the top pathways show little overlap between the four groups, which provides evidence for VIStA's ability to capture molecular elements that are specific to each subtype. Several identified pathways were related to metabolism, diabetes and inflammation. Group I was most enriched with inflammatory pathways including, for example, the FC-Gamma-R mediated phagocytosis (p=0.007) and CDC6-association with ORC:origin-complex pathways (p=0.15). Other pathways include small lung cancer (p=0.004) and maturity onset diabetes of the young (p=0.009) [15]. Group II was enriched with lipid transport and beta-cell and insulin signaling pathways like beta cell (p=0.005), HDL mediated lipid transport (p=0.006) and GTP hydrolysis pathways (p=0.007). In group III, pathways related to cell cycle control like mitotic prometaphase (p=0.0048), and downstream signaling pathways (p=0.003) with innate-immunity and GAB1 signaling were enriched. In group IV, distinct gap channel and inflammation pathways were identified like peptide ligand binding (p=0.0006), gap junction assembly (p=0.0008) and chemokine signaling pathways (p=0.0013).

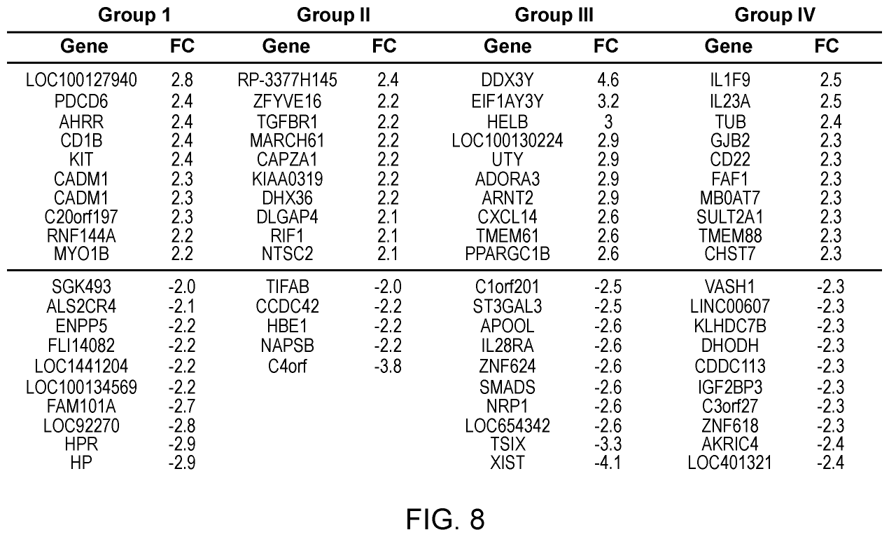

Genes with at least a 2-fold change (FC) in expression were identified at an FDR of <0.05, (see FIG. 8) for the specific set of up regulated and down regulated genes in each subgroup. For example, MMP7 was found to be up regulated in Group I (BMI). This result is consistent with findings in Maquoi et al., Diabetes, 51(4): 1093-1101 (2002), where nutritionally induced obese mice showed alterations in MMPs and TIMPs expression, thus providing further evidence for the role of these proteolytic system genes in COPD subtype with low BMI.

These results show that the VIStA assay is useful for identifying up regulated and down regulated genes within subgroups and identify active pathways of diseases.

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled. It is to be understood that this invention is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into sub-ranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. A method for determining at least one subgroup of subjects from a population of subjects diagnosed with the same disease comprising:
(A) obtaining the population of subjects diagnosed with the same disease;
(B) dividing the population of subjects into a first group and a second group;
(C) calculating the number of differentially expressed genes between the first group and the second group;
(D) exchanging one subject from the first group with one subject from the second group;
(E) re-calculating the number of differentially expressed genes between the first group and the second group, wherein the exchange of subjects between the first group and second group is maintained if the number of differentially expressed genes between the first group and the second group increases, and wherein the exchange of subjects is rejected if the number of differentially expressed genes between the first group and the second group remains the same or decreases; and
(F) repeating steps D-E.

2. The method of claim 1 further comprising identifying at least one clinical characteristic of the disease, wherein the at least one clinical characteristic is statistically significantly different between the first group and second group.

3. The method of claim 1, wherein the division of the population of subjects is random.

4. The method of claim 1, wherein determination or re-determination of the number of differentially expressed genes between the first group and the second group is determined by Significant Analysis of Microarrays (SAM), p-values of simple t-tests, Mann-Whitney U-test, Analysis of Variance (ANOVA), and minimal fold change.

5. The method of claim 2, wherein the statistically significant difference of the at least one clinical characteristic is determined by a Mann-Whitney U-test, Fisher's exact test, t-test, Analysis of Variance (ANOVA), chi-square test, Kolmogorov-Smirnov test.

6. The method of claim 1, wherein repeating steps D-E are performed about 1000 times.

7. The method of claim 2, wherein the statistical significant difference is measured as p≤0.05.

8. A method for identifying at least one subgroup of subjects from a population of subjects diagnosed with the same disease comprising:
(A) obtaining the population of subjects diagnosed with the same disease;
(B) dividing the population of subjects into a first group, a second group, and a third group;
(C) determining the number of differentially expressed genes between the first group and the second group;
(D) exchanging one subject from the first group with a first subject from the third group and one subject from the second group with a second subject from the third group;
(E) re-determining the number of differentially expressed genes between the first group and the second group, wherein the exchange of subjects between the first group, second group, and third group are maintained if the number of differentially expressed genes between the first group and the second group increases, and wherein the exchange of subjects between the first group, second group, and third group are rejected if the number of differentially expressed genes between the first group and the second group remains the same or decreases; and
(F) repeating steps D-E.

9. The method of claim 8 further comprising identifying at least one clinical characteristic of the disease, wherein the at least one clinical characteristic is statistically significantly different between the first group and second group.

10. The method of claim 8, wherein the division of the population of subjects is random.

11. The method of claim 8, wherein the determination or re-determination of the number of differentially expressed genes between the first group and the second group is determined by Significant Analysis of Microarrays (SAM), p-values of simple t-tests, Mann-Whitney U-test, Analysis of Variance (ANOVA), and minimal fold change.

12. The method of claim 9, wherein the statistically significant difference of the at least one clinical characteristic is determined by a Mann-Whitney U-test, Fisher's exact test, t-test, Analysis of Variance (ANOVA), chi-square test, Kolmogorov-Smirnov test.

13. The method of claim 9, wherein the statistical significant difference is measured as p≤0.05.

14. The method of claim 8, wherein repeating steps D-E are performed about 1000 times.

15. A method for identifying one or more clinical characteristics that identify a subgroup of subjects from a population of subjects diagnosed with the same disease comprising:
(A) obtaining the population of subjects diagnosed with the same disease;
(B) dividing the population of subjects into a first group and a second group;
(C) determining the number differentially expressed genes between the first group and the second group;
(D) exchanging one subject from the first group with one subject from the second group;
(E) re-determining the number of differentially expressed genes between the first group and the second group, wherein the exchange of subjects between the first group and second group is maintained if the number of differentially expressed genes between the first group and the second group increases, and wherein the exchange of subjects is rejected if the number of differentially expressed genes between the first group and the second group remains the same or decreases;
(F) repeating steps D-E; and
(G) identifying one or more clinical characteristics of the disease, wherein the clinical characteristics are statistically significantly different between the first group and second group.

16. The method of claim 15, wherein the division of the population of subjects is random.

17. The method of claim 15, wherein determination or re-determination of the number of differentially expressed genes between the first group and the second group is determined by Significant Analysis of Microarrays (SAM), p-values of simple t-tests, Mann-Whitney U-test, Analysis of Variance (ANOVA), and minimal fold change.

18. The method of claim 15, wherein the statistically significant difference of the at least one clinical characteristic is determined by a Mann-Whitney U-test, Fisher's exact test, t-test, Analysis of Variance (ANOVA), chi-square test, Kolmogorov-Smirnov test.

19. The method of any one of claims 1-18, wherein the disease is chronic obstructive pulmonary disease (COPD).

20. The method of any one of claim 2, 6, 9, 13, or 15, wherein the clinical characteristics of the disease are selected from the group consisting of chronic bronchitis, history of exacerbations, airflow limitation severity (GOLDCD), emphysema quantified by density mask analysis (FV950) or assessed qualitatively by a radiologist (EMPHETCD), body mass index (BMI), phlegm (PHLEGM), age (AGE), 6 minute distance walk (DWALK), cough (COUGH), and sex (SEX).
